(19) United States
(12) Patent Application Publication
(10) Pub. No.: US 2025/0152972 A1
(43) Pub. Date: May 15, 2025

(54) DIGITAL MODULATED RADIOGRAPHY

(71) Applicant: Virginia Commonwealth University, Richmond, VA (US)

(72) Inventors: Siyong Kim, Glen Allen, VA (US); Richard Wargo, Richmond, VA (US)

(21) Appl. No.: 18/924,246

(22) Filed: Oct. 23, 2024

Related U.S. Application Data
(60) Provisional application No. 63/545,271, filed on Oct. 23, 2023.

(51) Int. Cl. A61N 5/10; A61B 6/00; A61B 6/42; A61B 6/50; G06T 5/50; G06T 5/60; G06T 7/00; G06T 7/33; G16H 30/40

(52) U.S. Cl. CPC A61N 5/1049; A61B 6/4241; A61B 6/482; A61B 6/502; G06T 5/50; G06T 5/60; G06T 7/0016; G06T 7/337; G16H 30/40; A61N 2005/1062; G06T 2207/10124; G06T 2207/20081; G06T 2207/20084; G06T 2207/30068

(57) ABSTRACT

Digital modulated radiography improves radiographic image quality in the overall image and in sub-areas by enhancing image contrast in 2D/3D space. Devices and processes include a software-based approach using artificial intelligence (AI) to achieve energy modulation without the need for multiple exposures. By employing machine learning (ML) models, specifically Generative Adversarial Networks (GANs), images are translated between different energy domains, enhancing image contrast and potentially reducing radiation dose.

### CROSS-REFERENCE TO RELATED APPLICATIONS

This application claims the benefit of U.S. Provisional Patent Application No. 63/545,271, filed Oct. 23, 2023, the complete contents of which are herein incorporated by reference.

### FIELD OF THE INVENTION

Embodiments of this disclosure generally relates to radiography and associated medical practices and, in particular, techniques offering improved radiograph visualizations which can improve effectiveness of treatments and patient outcomes.

### BACKGROUND

Medical imaging is an essential tool in the diagnosis and treatment of various ailments and diseases, with image quality significantly impacting clinical outcomes. Traditional digital radiography often involves setting specific acquisition parameters such as automatic exposure control, image intensity, and x-ray energy, which attempt to optimize the image quality for the whole image but may not be ideal for all sub-regions.

In radiation therapy, patient setup verification is critical and often performed by comparing a reference image and a projection image at the time of radiation delivery. The quality and information conveyed by the reference image is therefore of great consequence.

### SUMMARY

Radiographic image quality depends on various factors including displayed image intensity (e.g., “window and level”), the amount of radiation used (i.e., radiation intensity), and the energy of the radiation beam. In conventional radiography, an image is obtained with a given set of those parameters based on the image task, anatomical makeup, and region of interest. Although such parameters are optimized over the whole image area or volume, they may not be necessarily optimal for every sub-region of the image. Exemplary devices and processes of this disclosure obtain an image which is optimized in sub-areas and/or sub-volumes by modulating one or more of the aforementioned three parameters in 2D/3D space.

Three modulation components which exemplary devices and methods may affect are 1) image intensity, 2) radiation intensity, and 3) radiation energy. Modulation for each component can be made as follows:
1) image intensity—by adjusting window and level,
2) radiation intensity—by multiple segmented radiation exposures varying the total radiation output with or without additional filters present, and
3) radiation energy—by either multiple segmented exposures with different energy beam(s) or generating artificial intelligence (AI) aided energy-modulated sub-area (or sub-volume) images. An exemplary digital modulated radiographic image is obtained by an optimal combination of image intensity, radiation intensity, and radiation energy. The digitally modulated content of an output image may be the whole image or just sub-area/ROIs within the image.

According to an aspect of some exemplary embodiments of this disclosure, a digital radiographic image (e.g., a digital x-ray image) is provided with single or multiple sub-areas or sub-volumes, each obtained with a different combination image intensity, radiation intensity and/or radiation energy. In this disclosure some exemplary processes having such an advantageous feature are referred to as “digital modulated radiography” or “modulated radiography”. Health care teams can obtain more information from modulated radiography compared to conventional radiography.

An exemplary generated image contains at least two parts which appear together (e.g., are displayed concurrently side-by-side) but differ from one another in subject contrast. In particular, the energy domain (e.g., energy bin, energy level) differs among the respective parts of the image. The parts are still appropriated regarded as belonging to the same image, rather than being distinctly separate images, based on their physical alignment with one another so that there is alignment/continuity of the anatomy portrayed by the image parts. Parts of an image which differ from one another in subject contrast may or may not also differ from one another in display contrast. “Subject contrast” and “display contrast” are distinctly different types of contrast in the context of radiography. The respective parts of an image which differ from one another in contrast (e.g., differ in at least subject contrast) may be referred to in this disclosure as parts, areas, volumes, and/or regions. In addition, these terms may be qualified with the “sub-” prefix to clarify that less than a whole/entirety of an image is being discussed. Those of skill in the art will recognize from context that “sub” may be implied without the prefix being expressly included. Parts of an image which adjoin one another may be referred to as “neighboring” one another.

A machine learning (i.e., artificial intelligence) model may be configured to translate an image or part thereof from one energy domain to some other energy domain. A model may be configured to, for example, start from a polyenergetic energy domain and translate to another polyenergetic domain and/or one or more monoenergetic domains. The energy domain of the translated image may be expressly characterized as “virtual” to clarify that it is obtained by a computer-implemented procedure rather than being the energy domain to which the subject (e.g., patient) was subjected during initial imaging (e.g., with a projectional imaging apparatus (e.g., xray, CT scanner, etc.)). Those of skill in the art will recognize the “virtual” qualifier may be implied based on context in this disclosure. A model configured for translation between/among energy domains may be trained on paired datasets representing subject (e.g., human or animal) anatomy at different energies. “Paired” in this context means two or more associated datasets representing the same subject. Training data may be real patient images, virtual/simulated images (e.g., of a digital phantom patient), or a combination of both. Once trained, an exemplary model is able to take as few as a single input image with a first energy domain and translate that image to a second/different energy domain. In a clinical setting, the input available for translation is going to be a polyenergetic x-ray acquisition, for example.

Modulated radiography helps users to interpret images more efficiently and effectively. Modulated radiography can be realized in different level with various combinations of the three components mentioned above. Exemplary embodiments are usable in various radiology procedures and image-guided radiation therapies. In view of the present disclosure, medical teams can obtain more effective medical images and interpret them better, resulting in improved patient care and patient outcomes.

In some embodiments, modulation may be achieved by adjustment of settings of imaging equipment. For instance, outside of using window and level techniques and varying radiation intensity for modulation, the radiation (e.g., x-ray) energy may be altered, directly affecting the contrast/image quality within an image upon capturing of the image. Energy modulation for sub-areas and/or sub-volumes can be made using segmented exposures with radiation beams in different energies. This approach may require additional hardware support and increases patient dose.

As an alternative to modulating image capture parameters, some embodiments achieve modulated radiography by a software-based approach. One example of an exemplary software solution is to generate synthetic energy modulated images, e.g., using machine learning and pre-existing imaging data obtained at different energies.

According to an aspect of some exemplary embodiments, a computer-implemented model is disclosed which is capable of translating images between different energy domains, thereby improving contrast. By acquiring an image at a specific polyenergetic energy and subsequently translating it to another polyenergetic or monoenergetic energy, overall image quality is enhanced. A non-limiting example application is creation of virtual monoenergetic images (e.g., 40-190 keV) or material-specific images from, e.g., dual-energy CT datasets.

A translated image may represent a different x-ray energy for an entire image or replace a region of interest (ROI) (or multiple ROIs) within the original image. An exemplary model for image-to-image translation in modulated radiographic imaging may, for example, leverage DECT datasets and corresponding virtual monoenergetic reconstructions.

Exemplary models may be any of various commercially available or custom machine learning models. As a non-limiting example, an exemplary model is a generative adversarial network (GAN). GANs may be used for image-to-image translation tasks. These translation tasks offer several advantages, including potential dose reduction, improved image contrast, shorter MRI examination time, and valuable information for radiotherapy planning. Generative adversarial networks (GAN) are a class of deep learning models with two main components or neural networks: a generator and a discriminator. The training process of GANs may be described as an adversarial game where the generator tries to create increasingly realistic data to fool the discriminator, while the discriminator becomes better at distinguishing between real and fake data. Various variables impact model performance. Exemplary embodiments may take into account, for example, variables ranging from the model options and hyperparameters to the input image characteristics (e.g., X-ray source spectrum, detector properties, image processing) for model use and initial training/testing.

Generative adversarial networks are particularly well-suited for addressing radiographic translation tasks given the availability of appropriate paired datasets. In some embodiments, a GAN uses paired image datasets representing the same anatomy at different energies. Alternatively, some embodiments may use digitally reconstructed radiographs (DRRs) from dual-energy CT (DECT) datasets and corresponding virtual monoenergetic reconstructions. This approach generates varying levels of contrast based on the polyenergetic and monoenergetic energies represented in the datasets.

Exemplary embodiments may include a conditional generative adversarial network (cGAN). An exemplary cGAN may comprise a generator and a discriminator, and the discriminator may be a patchGAN that evaluates one or more individual patches of input images instead of entireties of the input images. In this disclosure, the term “generator” is interchangeable with “generator network,” and the term “discriminator” is interchangeable with “discriminator network”.

In some embodiments, a model used for translating images between energy domains may first be trained on datasets coming from a single dual-energy CT technology.

To address the underdetermined nature of X-ray attenuation, some embodiments may incorporate physics-based regularization loss and/or additional dataset filtration, such as patient thickness, to improve model performance while maintaining generalizability. These steps may help the model to better differentiate between materials with similar attenuation characteristics, thereby enhancing the accuracy and robustness of the image translation.

Exemplary embodiments may involve or be employed in the context of any of a variety projectional imaging modalities, including but not limited to mammography and fluoroscopy. Modulated radiography has the potential to help users interpret images more efficiently and effectively for diagnostic purposes or for easier patient alignment in radiation therapy. For instance, acquiring an image at a higher polyenergetic level and translating it to a lower energy domain can improve inherent contrast and potentially reduce noise or lower doses. Depending on the embodiment, if desired, translation may alternatively be made from a lower energy domain to a higher energy domain.

The advent of photon counting detectors in clinical CT presents an opportunity to utilize paired datasets from photon counting detectors for training exemplary machine learning models for purposes of the technology of this disclosure. Photon counting detectors (PCDs) provide a unique opportunity for paired datasets for a plurality of various energy bins (e.g., low energy bin, mid energy bin, high energy bin). The associated datasets are all representative of the same subject and are acquired at the same time. According to some exemplary embodiments, datasets from one or more PCDs may be used to train a model, and then that model may be used to translate an acquired image which may be acquired by means other than a PCD. The model is able to simulate a PCD, providing one or more translated images at different energy levels. The model in essence can simulate the energy discrimination of a PCD, even when the acquired image is, for example, a traditional polyenergetic x-ray acquisition.

Diagnostic applications of exemplary embodiments can include, for example, any ordered radiographic/projectional x-ray imaging study by a physician. In general radiography, this can mean acquiring images at a higher energy (less dose to the patient) and then translating to a lower energy typically used for the study. Another application is providing a means to do dual-energy chest radiography in one x-ray acquisition. Yet another exemplary application is a built-in tool for radiologists to present the anatomy in a different way that may make their diagnoses easier. Exemplary embodiments may be used in mammography, enabling dual-energy mammography with a single X-ray acquisition. In this specific example, an exemplary embodiments provides the ability to quantify the amount of dense tissue/estimate the percentage of breast density, which can be tracked over time and/or give the clinician the ability to adjust his screening approach with additional imaging modalities (e.g., MRI, ultrasound).

Some exemplary embodiments which involve DRRs may include additional image enhancement through processing or other machine learning models to overcome the resolution mismatch between the input of conventional digital radiographs, which have superior resolution, and the DRRs that the models are trained on.

Some embodiments may comprise an application specific integrated circuit (ASIC) for an artificial neural network (ANN), the ASIC comprising: a plurality of neurons organized in an array, wherein each neuron comprises a register, a microprocessor, and at least one input; and a plurality of synaptic circuits, each synaptic circuit including a memory for storing a synaptic weight, wherein each neuron is connected to at least one other neuron via one of the plurality of synaptic circuits.

An Example below provides proof of concept for a digital modulated radiography framework, demonstrating the feasibility and effectiveness of integrating machine learning methods. The Example establishes a framework for translating between energy domains using digitally reconstructed radiographs (DRRs) from dual-energy CT datasets and derived monoenergetic reconstructions via Pix2Pix. DRRs were generated in 15° increments from 0° to 90° across different energy domains. There were 3,500 images in each energy domain (2 polyenergetic, 4 monoenergetic; 500 patients×7 angles=3,500 images). Training models translate between the polyenergetic domains and from polyenergetic to monoenergetic domains, as this approach is more representative of a potential clinical workflow. Preliminary testing involved hyperparameter tuning and model optimization, followed by training and testing on various dataset splits, including cross-validation and projection-specific datasets. Quantitative metrics (PSNR, SSIM, MSE, MAPE) and qualitative analysis (visual inspection of difference maps) were used to assess the various model performance. The models trained using cross-validation on the various energy translations produced the following results: PSNR: 29.1±2.0, SSIM: 0.947±0.017, MSE: 169.1±68.3, MAPE: 8.2%±1.8%. In contrast, the models trained using cross-validation to translate between the polyenergetic high energy and polyenergetic low energy on the projection-specific datasets (anterior-posterior [0°] and lateral views [90°]) achieved the following results: PSNR: 27.4±0.5, SSIM: 0.909±0.003, MSE: 195.9±39.7,MAPE: 10.4%±2.1%.

### BRIEF DESCRIPTION OF THE DRAWINGS

FIG. 1A is an exemplary process of digital modulated radiography and subsequent actions.

FIG. 1B is a block diagram of exemplary hardware involved with the execution of some exemplary processes.

FIG. 2 is a planar modulated radiographic image with sub-areas which differ from other areas of the overall image in one or more of image intensity, radiation intensity, and radiation energy.

FIG. 3 is an example of modulated radiography that includes a sub-area for which the chosen image intensity (i.e., window and level) set is different from the image intensity set of the main image.

FIGS. 4A-4D are different radiographs for patient setup in breast radiation therapy.

FIG. 5 is an exemplary process for data preparation, training, and model deployment for radiation therapy applications.

FIG. 6 shows an initial polyhigh image (left) and a modulated image (right) based on the initial polyhigh image. The modulated image includes, in part, the initial polyhigh image and further includes a generated 60 keV region-of-interest (ROI) overlay.

FIG. 7 shows DRR processing for (top) maximum and (bottom) minimum cases. Images were obtained from the 120 keV energy domain, and the projection shown is AP/0°. The full DRRs were cropped and centered to 512×512 images. Subsequently, they were processed in two ways: (1) split into 256×256 quadrants (UL, UR, LL, LR), or (2) cropped and centered into 256×256 images.

FIG. 8A shows best-performing and worst-performing cross-validation trained models on two different energy domain translations using the SSIM defined quantitative metric as a function of epochs.

FIG. 8B shows best-performing and worst-performing cross-validation trained models on two different energy domain translations using the PSNR defined quantitative metric as a function of epochs.

FIG. 8C shows best-performing and worst-performing cross-validation trained models on two different energy domain translations using the MSE defined quantitative metric as a function of epochs.

FIG. 8D shows best-performing and worst-performing cross-validation trained models on two different energy domain translations using the MAPE defined quantitative metric as a function of epochs.

FIGS. 9A and 9B are images associated with the minimum, median, and maximum results for SSIM, PSNR, and MAPE for the worst performing model energy domain translations at the 300th epoch. For reference, the input image, the corresponding image at the 5th epoch, and the ground truth image are also provided.

FIG. 9C and 9D are images associated with the minimum, median, and maximum results for SSIM, PSNR, and MAPE for the best performing model energy domain translations at the 300th epoch. For reference, the input image, the corresponding image at the 5th epoch, and the ground truth image are also provided.

### DETAILED DESCRIPTION

Image quality depends on various factors including displayed image intensity (e.g., window and level), the amount of radiation used (i.e., radiation intensity), and the energy of the radiation beam. “Displayed contrast” within an image can be adjusted using window and level settings, which control the range and midpoint of pixel values displayed on the monitor. A wider window shows more shades of gray, while adjusting the level changes the brightness. In digital radiography and mammography, “for processing” images are raw or minimally processed images, often corrected for dead pixels, noise, and other artifacts. “For presentation” images undergo additional adjustments for diagnostic purposes, such as contrast enhancement and image sharpening. “Subject contrast” refers to the fundamental contrast arising in the signal after interacting with the patient but before detection, influenced by intrinsic (anatomical) and extrinsic (x-ray energy) factors. Some exemplary embodiments of this disclosure focus on modifying subject contrast. For instance, some exemplary embodiments of this disclosure focus on modifying subject contrast dependent on extrinsic factors by altering the x-ray energy.

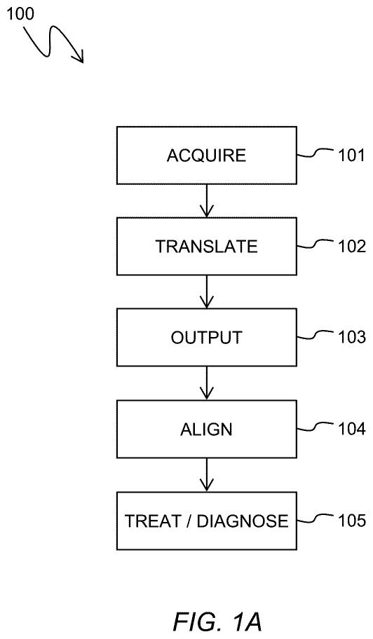

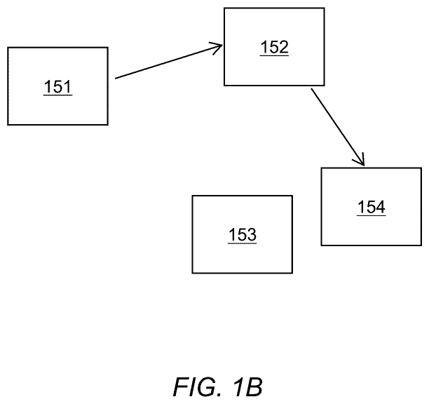

FIG. 1A is an exemplary process 100 of digital modulated radiography and subsequent alignment of a subject (e.g., patient) who is to be subjected to radiation energy or for diagnostic purposes. The exemplary process 100 includes but is not limited to the following steps. Step 101 is acquiring an image at a first energy domain, e.g., a first polyenergetic energy. “Acquiring” as used here may refer to taking a new radiographic image of a patient, e.g., by known procedures for x-ray, CT scan, mammography (mammogram), fluoroscopy, or other radiography technique. In addition or in the alternative, “acquiring” may refer to loading of a radiographic image (i.e., image data) into a computer, e.g., from memory. Step 102 is translating at least part of the acquired image to a translated image of another energy domain different from the first energy domain. For instance, step 102 may translate an image or part thereof from a first polyenergetic energy to some other polyenergetic or monoenergetic energy. The translated image may be referred to as a virtual reconstruction in some embodiments. Step 103 is outputting a generated image of the subject in which at least one region of interest (ROI) which is a sub-area and/or sub-volume of the generated image is based on the translated image. The output image may be referred to as a modulated image. The subpart (i.e., the sub-area and/or sub-volume) of the generated image may be a subpart of the translated image, with or without additional processing of the translated image before the translated image is used for content of the sub-area and/or sub-volume of the generated image. In this discussion, “sub-” means less than an entirety, i.e., less than the whole. The sub-area and/or sub-volume may be characterized as an inset, window, and/or overlay in some embodiments. Up to all of the respective parts of a generated image may each be based on a translated image (the same translated image, or different translated images). Alternatively, at least one part of the generated image may be based on a translated image, but some other part of the generated image may correspond with the acquired image (pre-translation).

The at least one region of interest (ROI) of the output step 103 exhibits a difference (which may visually appear as a discontinuity), e.g. of subject contrast, with a neighboring region of the generated image. The difference/discontinuity of subject contrast derives from (i.e., is based on) a difference in one or more of image intensity, radiation intensity, and radiation energy. Yet the at least one ROI is in semantic context with the neighboring region of the generated image. For example, the at least one ROI exhibits continuity of subject anatomy with the neighboring region. One or more contours or edges in the ROI align with corresponding contours or edges in the neighboring region. The at least one ROI (the sub-area or sub-volume) is configured to match surroundings areas of the generated image in scale and perspective, for example.

“Outputting” may include one or more of sending to long-term memory, recording in long-term memory, retrieving from memory, and displaying the output with a display device such as a monitor, display, smartphone, projector, or the like.

Step 104 is aligning (e.g., setting up) a patient using the image generated by step 103. Alignment is made with respect to radiation therapy equipment, for example. Step 104 may be or include performing image registrations with the modulate image(s) as reference images. After alignment is completed, the aligned subject is subjected to treatment, e.g., to radiation energy from the radiation therapy equipment (radiation therapy), at step 105. An image generated by step 103 may be used for other purposes besides alignment preceding radiation therapy. For example, the image generated by step 103 may be used for one or more diagnostic purposes.

One or more aspects of exemplary process 100 may be computer-implemented, e.g., performed by one or more processors of computers, servers, etc. In particular, steps 101, 102, and 103 or any subgrouping thereof may be entirely computer-implemented as a method of generating an image, in particular, a digital modulated radiograph.

FIG. 1B is a block diagram of exemplary hardware suited to the execution of some exemplary processes of this disclosure. Imaging equipment 151 is, for example, a CT scanner. Computer(s) 152 may be one or more standalone computers (e.g., servers) and/or be special purpose computers and/or on-board computing hardware of other equipment shown in FIG. 1B. Therapy equipment 153 is, for example, a linear accelerator (LINC). Output device(s) 154 is, for example, a digital screen (e.g., of a smartphone, tablet, PC, etc.), a projector, a virtual reality (VR) device, and/or an augmented reality (AR) device. Following is a non-limiting example of an exemplary method employing the indicated hardware. Computing device(s) 152 create/generate a modulated image from initial image data. Devices 152 may be, for example, one or more computers, processors (e.g., image processor), etc. The initial image data may be, for example, “for processing” images originating from imaging equipment 151. The modulated image data is sent to one or more devices 154, which may be or include, for example, an augmented reality (AR) device or some other display device. A user such as a doctor, nurse, or technician at a medical facility may use the device 154 for setup of a patient and therapy equipment 153 prior to initiating the energy delivery of the radiation therapy and/or during the course of the radiation therapy. The device 154 may, in some implementations, be configured to receive user input such as but not limited to hand gestures or voice commands to adjust which modulated image is actively shown by the device 154 or to adjust the modulated image or to cause some other change to the content display by device 154.

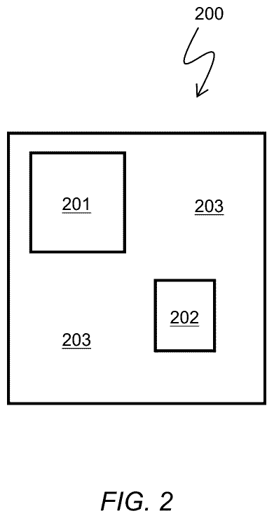

FIG. 2 is a simplified depiction of a modulated radiographic image 200. The image 200 consists of sub-areas 201 and 202 and neighboring region(s) 203. The sub-areas 201 and 202 are each obtained in a combination of different image intensity, radiation intensity, and radiation energy.

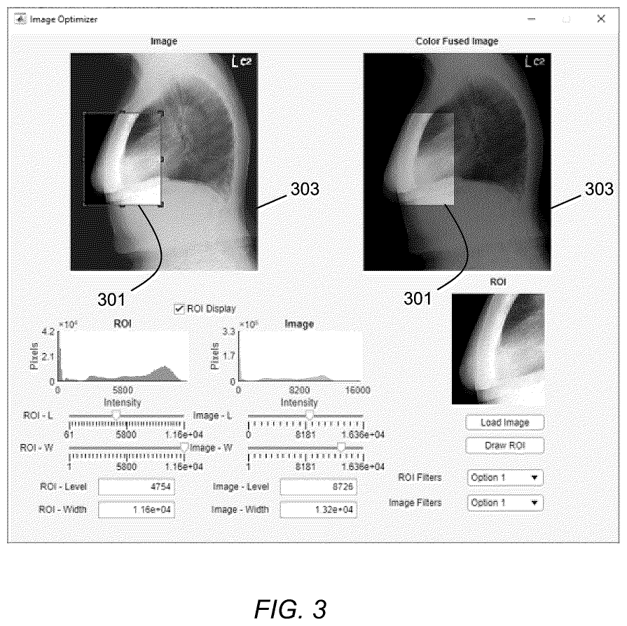

FIG. 3 illustrates a simple example that includes a sub-area 301 where the image intensity (i.e., window and level) set chosen is different from what used for the main image 303. In this instance, display contrast has been changed for the sub-area relative to the surrounding area.

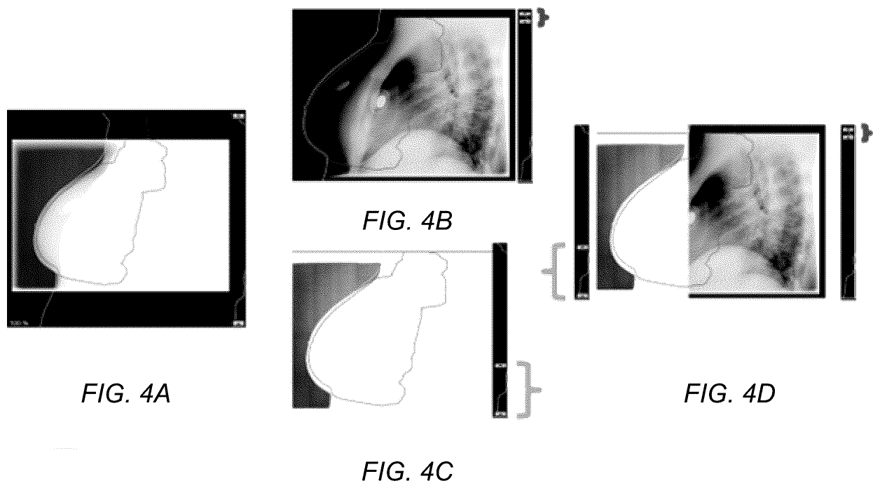

FIGS. 4A-4D together offer another example that illustrates how modulated radiography can be utilized in breast radiation therapy. For accurate treatment, both breast outline and chest wall (e.g., rib cages) should be aligned properly.

In conventional practice, planar images are typically obtained, and image registrations are performed with the reference images. The problem is that typical reference images show either the breast tissue well (at the cost of clear depiction of the chest wall) or the chest wall well (at the cost of clear depiction of the breast tissue). FIG. 4A is an example of a lateral image in full window display where bones are invisible and breast outline is visible but still unclear. Bone visibility can be enhanced by adjusting window & level as shown in FIG. 4B. However, breast outline visibility is totally lost in FIG. 4B. FIG. 4C shows an image where the situation is opposite, that is, breast outline visibility is maximized but bone visibility is totally lost. Radiation therapy team carries out such processes back and forth to assure both bone and breast outline are aligned properly. This procedure is, obviously, time consuming and from time to time suboptimal because the operators cannot see the other part when they work on one part. This limitation is overcome by using modulated radiography as illustrated in FIG. 4D.

FIG. 4D is an exemplary digital modulated radiograph in which the full image is subdivided in two sub-areas. Each sub-area is displayed with its own optimal set of window & level which differs from the window and level of the other sub-area. As illustrated in FIG. 4D, both breast outline and bones are visible and clear in a single display.

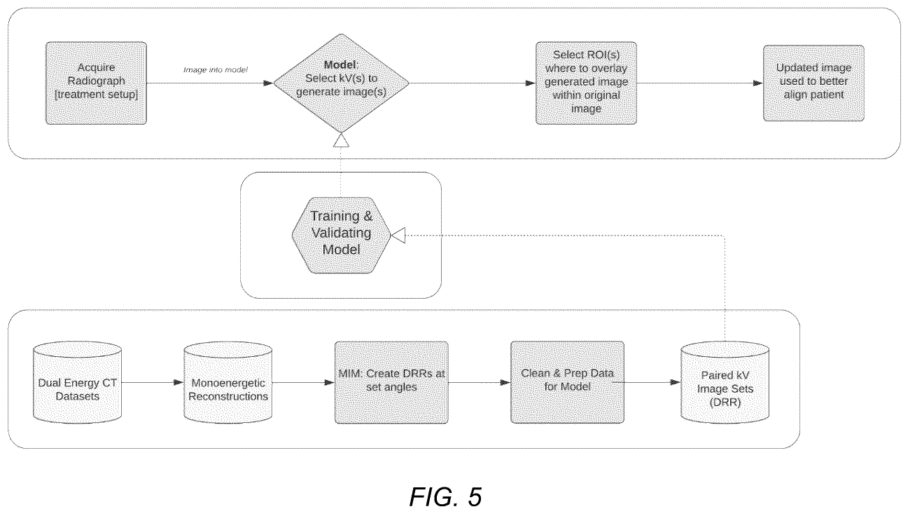

FIG. 5 illustrates an example where, using dual energy CT datasets, monoenergetic reconstructions are made to create digitally reconstructed radiographs for training models to translate images acquired at a given poly-energetic energy domain to other poly-energetic and monoenergetic energy domains for the purpose of accurate patient set up in radiation therapy.

According to some embodiments, an exemplary model comprises a generative adversarial network (GAN). Generative adversarial networks (GAN) are a class of deep learning models with two main components or neural networks: a generator and a discriminator. These networks are configured to play a game against each other during the training process. The generator's role is to create data, such as images, that should mimic real data samples. It takes random noise as input and transforms it into data representing the task at hand. The discriminator acts as an expert evaluating the data generated. The discriminator determines whether the data is real or fake data. In terms of images, the discriminator determines if the image is real or synthetic.

During the training process of an exemplary GAN, the generator tries to create increasingly realistic data to fool the discriminator, while the discriminator becomes better at distinguishing between real and fake data. This adversarial process pushes both networks to improve over time. The discriminator's feedback guides the generator to improve its output-making it more realistic over time. This iterative process continues until the generator becomes proficient at generating data that closely resembles real data, to the point where the discriminator has difficulty determining whether the sample is real or fake. At this stage, the generator has successfully captured the essential patterns and characteristics of the real data distributions, enabling it to produce new samples that closely match the features of the original data.

Within GANs, there are models based on supervised versus unsupervised learning which are differentiated by the type of data and the way the model is trained. Supervised learning in GANs refers to the scenario where the GAN model is trained using paired data, consisting of both input samples and their corresponding output samples. The generator learns to map input data to specific target outputs, guided by the provided pairs during training.

It is particularly advantageous for some embodiments that the model comprise a conditional generative adversarial network (cGAN). cGANs take random noise and conditional information as input. The extra information allows the generator to produce output data that is tailored to match the given conditioning. In the context of exemplary embodiments, the conditional information represents radiographic images acquired at particular polyenergetic energies or virtual monoenergetic energies. The discriminator also receives the same conditional information in addition to the input data. The additional information enables the discriminator to assess the realism of the generated data with regard to the given condition.

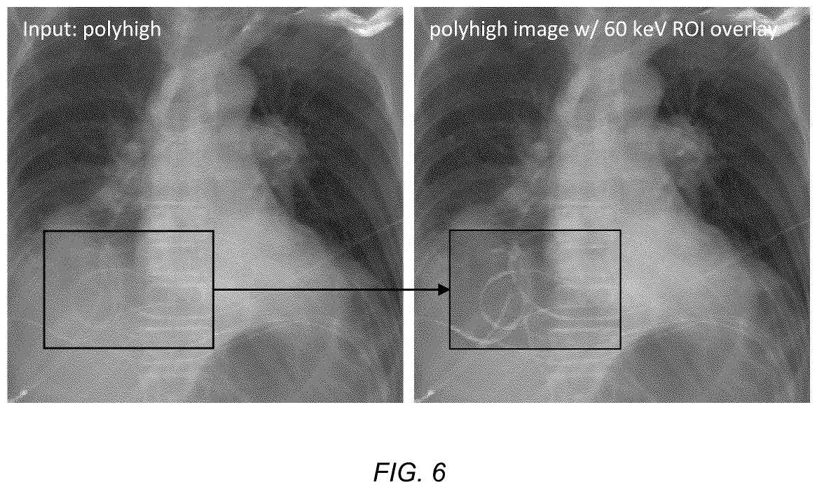

Another example of how the technology of this disclosure may be utilized is shown in FIG. 6. In FIG. 6, an area within the input image (figure left) is replaced with the corresponding area from a generated image at a particular energy with improved contrast. Figure right of FIG. 6 shows the composite generated image containing a combination of the input image together with a sub-area the contents of which are from the generated image at a particular energy different from the input image energy. The sub-area has improved contrast for a region of interest (ROI). Note that modulated radiography not only has the potential to generate multiple energy-modulated images but also could be utilized in a process where an optimized image has multiple sub-areas that have been replaced with the corresponding high-contrast sub-areas from the generated energy-modulated images.

In some embodiments, exemplary processes may include outputting one or more outputs such as generated/modulated images using one or more mediated reality devices. “Mediated reality” comprises one or more stimuli (e.g., visual content output or outputtable with a display device) by which reality is modified (e.g., diminished or augmented), often by a computer. The general intent is to enhance one's natural perception of reality (e.g., as perceived by their senses without external devices). A user experiences both real content and virtual content when experiencing mediated reality. In this disclosure, the expression “augmented reality” (“AR”) may be used synonymously with “mediated reality”.

AR may comprise active content and/or passive content. Active content may be, for example, a visual output on a display device or an auditory output on a speakerphone device. Passive content may be, for example, visual stimuli from natural surroundings. For instance, on a see-through head mounted display (HMD), the real world is naturally visible to a user through a see-through display surface of the device. Therefore a see-through HMD need only actively display virtual augmentations in order to provide AR content. Real world content is provided but is, in essence, provided passively. Real world content may be provided actively, by for example, capturing real world content with a camera and subsequently displaying the content (e.g., on a screen). The virtual content may be supplied as overlays or otherwise imbedded with the real world video content.

“Virtual reality” replaces the real world with a simulated one. If a system, device, or method results in a user experience that contains only virtual content (i.e., no real content), such result may be called “virtual reality” or “VR”.

In general, AR and VR outputs according to exemplary embodiments may take any of a variety of perspectives, including third-person, first-person, top-down, aerial, elevated, others, or some combination of these.

An “augmentation” is a unit of virtual content and may be, for example, a virtual object rendered as a graphic on a display device. An augmentation may be visual. In particular, some exemplary embodiments may provide one or more modulated radiograph images as an augmentation via a suitable output device. An “output device”, as used herein, may be a device capable of providing at least visual, audio, audiovisual, or tactile output to a user such that the user can perceive the output using his senses (e.g., using her eyes and/or ears). In many embodiments, an output device will comprise at least one display, at least one speaker, or some combination of display(s) and speaker(s). A suitable display (i.e., display device) is a screen of a mobile electronic device (e.g., phone, smartphone, GPS device, laptop, tablet, smartwatch, etc.). Another suitable output device is a head-mounted display (HMD). In some embodiments, the display device is a see-through HMD. In such cases the display device passively permits viewing of the real world without reproducing details of a captured real world image feed on a screen. In a see-through HMD, it is generally only the augmentations that are actively shown or output by the device. Visual augmentations are in any case superimposed on the direct view of the real world environment, without necessarily involving the display of any of the original video input to the system. In fact, for systems which do not use the video input to detect image data, the system may include one or more HMDs that have no camera at all, relying entirely on other sensors (e.g. GPS, gyro, compass) to determine the relevant augmentations, and displaying them on otherwise transparent glasses or visors. Output devices and viewing devices may include or be accompanied by one or more input devices (e.g., buttons, speakers, motion sensors, touchscreens, menus, keyboards, data ports, etc.) for receiving user inputs.

Some embodiments of the present invention may be a system, a device, a method, and/or a computer program product. A system, device, or computer program product may include a computer readable storage medium (or media) having computer readable program instructions thereon for causing a processor to carry out aspects of the present invention, e.g., processes or parts of processes or a combination of processes described herein.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Processes described herein, or steps thereof, may be embodied in computer readable program instructions which may be paired with or downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Java, Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions and in various combinations.

These computer readable program instructions may be provided to one or more processors of one or more general purpose computers, special purpose computers, or other programmable data processing apparatuses to produce a machine or system, such that the instructions, which execute via the processor(s) of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Where a range of values is provided in this disclosure, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are described.

As used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely”, “only”, and the like in connection with the recitation of claim elements, or use of a “negative” limitation.

As will be apparent to those of skill in the art upon reading this disclosure, each of the individual embodiments described and illustrated herein has discrete components and features which may be separated from or combined with the features of any of the other several embodiments without departing from the scope or spirit of the present invention. Any recited method can be carried out in the order of events recited or in any other order which is logically possible.

### EXAMPLE: AN ENERGY MODULATION FRAMEWORK FOR PROJECTIONAL MODULATED RADIOGRAPHY: PROOF OF CONCEPT USING DIGITALLY RECONSTRUCTED RADIOGRAPHS FROM DUAL-ENERGY COMPUTED TOMOGRAPHY AND A CONDITIONAL GAN

### 1|Summary

This Example presents an evaluation of machine learning to establish a framework for projectional modulated radiography. The Example shows effective utilization of dual-energy CT datasets and their subsequent monoenergetic reconstructions to construct DRRs for training models in the translation of images from one polyenergetic energy domain to another. The Example adapts existing image-to-image translation models, particularly Pix2Pix. The models show significant results across various performance metrics, highlighting the robustness and potential of the disclosed approach.

### 2|Materials and Methods

### 2.1 Datasets

This Example uses dual-energy (DE) CT datasets from 500 patients with pulmonary embolism cases. The DE studies were performed on Siemens CT scanners using dual-source technology or through a single source using Siemens TwinBeam technology. The dual-source technology uses two independent x-ray sources operated at two different voltages with the higher energy spectrum using a tin spectrum for even better spectral separation. Siemens TwinBeam technology uses a single source operating at a given voltage (120 kVp or 140 kVp) filtered by either tin (high energy) or gold (low energy) to generate spectral separation.

Of the 500 patients, 428 were acquired with the dual-source CT and 72 with the CT using DE TwinBeam. A more detailed breakdown of the initial CT datasets and DRR datasets can be found in Table 1.

TABLE 1
CT and DRR dataset Information.

| | |
|---|---|
| Parameter | Value(s)/information |

CT Dataset information

| | | |
|---|---|---|
| Dual-energy | Dual Source (ds) | 428 [Scan FOV: 50 cm (A); |
| acquisition type | | 33 cm (B)] |
| | TwinBeam (tb) | 72 [Scan FOV: 50 cm] |

| Energies Represented | Polyenergetic (kVp): 100, |
|---|---|
| | 120(Au, Sn), 140(Au, Sn), |
| | 140(Sn) |
| | Monoenergetic (keV): 60, 80, |
| | 100, 120 |
| Recon Kernel | Bf37f, Br34f, Qr40f, Qr40f\3 |
| Recon FOV (mm) [min, mean +/− SD, max] | [263, 341 ± 34, 492] |
| Isocenter (mm) | 535, 595 |
| Source-to-detector distance (mm) | 976, 1085.6 |
| Pixel Size (mm) [min, mean +/− SD, max] | [0.51, 0.67 ± 0.07, 0.96] |
| Slice Thickness (mm) | 0.6 |
| Slice Interval (mm) | 0.4 |

DRR dataset information

| Energies Represented | Polyenergetic (kVp): 100, 120(Au, Sn), |
|---|---|
| | 140(Au, Sn), 140(Sn) |
| | Monoenergetic (keV): 60, 80, 100, 120 |
| DRR calculation method | Parallel Ray Algorithm |
| Source-to-detector distance (mm) | ∞ |
| Pixel size x (mm) | 1.0 |
| Pixel size y (mm) | 1.0 |
| Image size - rows (pixels) | [512, 755 ± 85, 1105] |
| [min, mean +/− SD, max] | |
| Image size - columns (pixels) | [512, 512 ± 0, 512] |
| [min, mean +/− SD, max] | |

The dual-energy CT datasets were processed on Siemens syngo. via which is a multimodality reading solution built on a client-server platform. It has many packages available for different imaging applications. One of the applications is making use of dual-energy CT datasets where virtual monoenergetic images [40-190 keV possible] or material-specific images can be created. Monoenergetic reconstructions were created at 60, 80, 100, and 120 keV. The 6 CT datasets [high polyenergetic, low polyenergetic, 60 keV, 80 keV, 100 keV, 120 keV] per patient were then anonymized and exported for further processing. The high polyenergetic (polyhigh) dataset includes the dual source 140 kVp and TwinBeam 120/140 kVp (Sn) data. The low polyenergetic (polylow) dataset includes the dual source 100 kVp and TwinBeam 120/140 kVp (Au) data.

The anonymized datasets were then uploaded into MIM Maestro which is a software package with a comprehensive set of radiation oncology tools. MIM was used for its ability to create digitally reconstructed radiographs (DRR) and workflows for streamlined processing. DRRs generated on-the-fly (outside of treatment plans) are created using parallel rays where the source-to-image distance is infinite as opposed to a virtual x-ray source with an image plane at a set distance. The pixel sizes (x,y) are hard-coded to be 1 mm. DRRs were generated for each dataset in 15° increments from 0° to 90°. This created 3,500 images in each energy domain (total domains: 2 polyenergetic, 4 monoenergetic) [500 patients×7 angles =3,500 images], totaling 21,000 images [3,500×6 energy domains=21,000 images].

### 2.2 Datasets Preparation

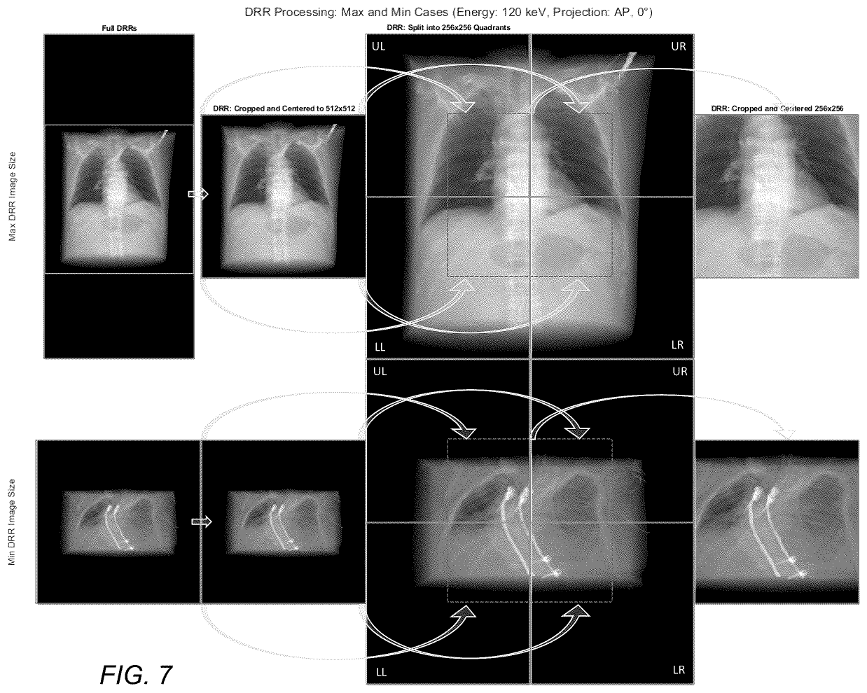

Further image processing and data analysis was performed in MATLAB. Due to requirements of the open-source iteration of Pix2Pix used on GitHub, the images were converted from DICOM format to portable network graphics format (png) and from 16-bit to 8-bit and RGB format. In order to go from grayscale to RGB images, all channels were set to the same pixel value for a given pixel location. There was also a restriction on the size of the images that could be used. The images ultimately were converted to the size restriction of 256×256 in a few different approaches. From Table 1, the maximum and minimum sizes of the DRRs were 1105×512 and 512×512 respectively [row, column]. The images were first cropped and centered to 512×512. From there, they were (1) split into 256×256 quadrants (UL=upper left, UR=upper right, LL=lower left, LR=lower right) or (2) cropped and centered again to 256×256. The described procedures can be visualized for the two extrema mentioned in FIG. 7. Conversion from 16-bit to 8-bit can be expressed as follows:

$${DRR}_{16 - bit , new} ( x , y ) = {DRR}_{16 - bit , orig} ( x , y ) + 1 \qquad ( 1 )$$

$${DRR}_{NORM} ( x , y ) = \frac{{DRR}_{16 - bit , new} ( x , y )}{max [ {DRR}_{16 - bit , new} ( x , y ) ]} \qquad ( 2 )$$

$${DRR}_{8 - bit} ( x , y ) = uint 8 ( ( {DRR}_{NORM} ( x , y ) \times {2}^{8} ) - 1 ) \qquad ( 3 )$$

### 2.3 Models

Pix2Pix is a deep-learning framework for image-to-image translation tasks. The mapping is learned in a supervised manner, meaning that paired input-output image datasets are required for training. The architecture of Pix2Pix is based on U-Net, which is a type of convolutional neural network (CNN) commonly used in image segmentation tasks. The U-Net consists of an encoder path that reduces the spatial dimensions of the input and a decoder path that upsamples the features back to the original resolution. There are also skip connections that connect the corresponding layers of the encoder and decoder. The skip connections help preserve low-level image details during the translation process.

Another aspect of Pix2Pix is that it is a conditional GAN (cGAN). cGANs take random noise and conditional information as input. The extra information allows the generator to produce output data that is tailored to match the given conditioning. In this Example, the conditional information represents radiographic images acquired at particular polyenergetic energies or virtual monoenergetic energies. The discriminator also receives the same conditional information in addition to the input data. The additional information enables the discriminator to assess the realism of the generated data with regard to the given condition.

A 256 U-net, which refers to the input image size that the U-net model is designed to handle, was used for the generator and a patchGAN (70x70) was used for the discriminator. PatchGANs are designed to evaluate individual patches of the input images instead of the whole image. A summary of both networks can be found in Table 2. The output is a grid of binary values and each element in the grid represents the discriminator's classification decision for a specific patch within the input image. They are still based on CNN architecture where the CNN slides over the input image in a convolutional manner. This allows the patchGAN to capture local image features effectively. The localized approach helps the discriminator provide more detailed feedback to the generator which in turn allows for better discrimination between real and fake patches. Ultimately, the generator benefits by learning to produce more realistic outputs at a finer spatial level.

TABLE 2
Pix2Pix Network Summaries for Generator and Discriminator.

| | |
|---|---|
| Layer (type) & Layer Numbers | Output Shape |

Generator Network (UNet - 256) Summary

| | | | |
|---|---|---|---|
| Conv2d, LeakyReLU | (1-2) | Unet - | [64, 128, 128] |
| Conv2d, InstanceNorm2d, | (3-5) | Encoder | [128, 64, 64] |
| LeakyReLU | (6-8) | | [256, 32, 32] |
| | (9-11) | | [512, 16, 16] |
| | (12-14) | | [512, 8, 8] |
| | (15-17) | | [512, 4, 4] |
| | (18-20) | | [512, 2, 2] |
| Conv2d, ReLU | (21-22) | Unet - | [512, 1, 1] |
| ConvTranspose2d, InstanceNorm2d | (23-24) | Decoder | [512, 2, 2] |
| UnetSkipConnectionBlock, ReLU | (25-26) | | [1024, 2, 2] |
| ConvTranspose2d, InstanceNorm2d, Dropout | (27-29) | | [512, 4, 4] |
| UnetSkipConnectionBlock, ReLU | (30-31) | | [1024, 4, 4] |
| ConvTranspose2d, InstanceNorm2d, Dropout | (32-34) | | [512, 8, 8] |
| UnetSkipConnectionBlock, ReLU | (35-36) | | [1024, 8, 8] |
| ConvTranspose2d, InstanceNorm2d, Dropout | (37-39) | | [512, 16, 16] |
| UnetSkipConnectionBlock, ReLU | (40-41) | | [1024, 16, 16] |
| ConvTranspose2d, InstanceNorm2d | (42-43) | | [256, 32, 32] |
| UnetSkipConnectionBlock, ReLU | (44-55) | | [512, 32, 32] |
| ConvTranspose2d, InstanceNorm2d | (46-47) | | [128, 64, 64] |
| UnetSkipConnectionBlock, ReLU | (48-49) | | [256, 64, 64] |
| ConvTranspose2d, InstanceNorm2d | (50-51) | | [64, 128, 128] |
| UnetSkipConnectionBlock, ReLU | (52-53) | | [128, 128, 128] |
| ConvTranspose2d, Tanh, UnetSkipConnectionBlock | (54-56) | | [3, 256, 256] |

Discriminator Network (PatchGAN 70 × 70) Summary

| | | |
|---|---|---|
| Conv2d, LeakyReLU | (1-2) | [64, 128, 128] |
| | (3-5) | [128, 64, 64] |
| Conv2d, InstanceNorm2d, | (6-8) | [256, 32, 32] |
| LeakyReLU | (9-11) | [512, 31, 31] |
| Conv2d | (12) | [1, 30, 30] |

Abbreviations:
Conv2d (2D convolution),
InstanceNorm2d (instance normalization),
LeakyReLU (leaky rectified linear unit),
ReLU (rectified linear unit),
ConvTranspose2d (transposed convolution),
UnetSkipConnectionBlock (UNet skip connection block),
Dropout (regularization),
and Tanh (hyperbolic tangent activation function)

### 2.4 Quantitative Analysis

Various image quality measures: peak signal-to-noise ratio (PSNR), structural similarity index measure (SSIM), mean squared error (MSE), and mean absolute percentage error (MAPE) were used to evaluate a models performance at various epochs. Additionally, visual assessment of SSIM maps and difference maps were used to assess the models qualitatively. The following metrics are calculated on an image-by-image basis within a test dataset for the generated and reference image. The results are then subsequently averaged to get a mean performance for a model on a given test dataset at a specific epoch.

PSNR is a commonly used metric in image processing to quantify the quality of a reconstructed image compared to the original, reference image.

$$PSNR = 10 \cdot {log}_{10} \frac{{MAX}_{I}^{2}}{MSE} ; \qquad ( 4 )$$

MAX_{I} is the maximum possible pixel value of the image. For 8-bit images, this corresponds to a value of 255 and 65535 for 16-bit images. MSE is the mean squared error and is defined below. SSIM is a widely used metric in image processing. It measures the similarity between two images by taking three components into consideration: 1) Luminance Comparison (l): evaluates similarity in brightness between images (based on contrast and brightness of the pixel values in both images), 2) Contrast Comparison (c): looks at the similarity in contrast between images, indicating image sharpness, and 3) Structure Comparison(s): quantifies the structural similarity between images showing how well the patterns and structures between images match.

$$l ( x , y ) = \frac{2 {μ}_{x} {μ}_{y} + {C}_{1}}{{μ}_{x}^{2} + {μ}_{y}^{2} + {C}_{1}} \qquad ( 5 )$$

$$c ( x , y ) = \frac{2 {σ}_{x} {σ}_{y} + {C}_{2}}{{σ}_{x}^{2} + {σ}_{y}^{2} + {C}_{2}} \qquad ( 6 )$$

$$s ( x , y ) = \frac{{σ}_{xy} + {C}_{3}}{{σ}_{x} {σ}_{y} + {C}_{3}} \qquad ( 7 )$$

$$SSIM ( x , y ) = {l ( x , y )}^{α} \cdot {c ( x , y )}^{β} \cdot {s ( x , y )}^{γ} = \frac{( 2 {μ}_{x} {μ}_{y} + {C}_{1} ) ( 2 {σ}_{xy} + {C}_{2} )}{( {μ}_{x}^{2} + {μ}_{y}^{2} + {C}_{1} ) ( {σ}_{x}^{2} + {σ}_{y}^{2} + {C}_{2} )} \qquad ( 8 )$$

$${C}_{1} = {( {k}_{1} L )}^{2} ; {C}_{2} = {( {k}_{2} L )}^{2} ; {C}_{3} = \frac{{C}_{2}}{2} \qquad ( 9 )$$

With α, β, γ set to 1, the formula reduces to the equation above. μₓ, μ_{y}, σₓ, σ_{y}, and σ_{xy} are the local means, standard deviations, and cross-covariance for images x, y. C₁ and C₂ are used to stabilize the division with a weak denominator. L is the dynamic range of the pixel values within the image. k₁=0.01 and k₂=0.03 by default. The SSIM value ranges from −1 to 1, where values closer to 1 indicate higher similarity between the images, with 1 implying an exact match. MSE measures the average squared difference between the generated image and the reference image. It is often used in machine learning and image processing to evaluate the performance of models or to quantify the accuracy of a model.

$$MSE = \frac{1}{mn} {\sum }_{i = 0}^{m - 1} {{\sum }_{j = 0}^{n - 1} [ I ( i , j ) - K ( i , j ) ]}^{2} \qquad ( 10 )$$

I is the m×n reference image where K is the m×n generated image.

MAPE is another means to measure the accuracy of a model. It quantifies the average percentage difference between the pixels within the generated and reference images.

$$MAPE = \frac{1}{mn} {\sum }_{i = 0}^{m - 1} {\sum }_{j = 0}^{n - 1} \vert \frac{I ( i , j ) - K ( i , j )}{I ( i , j )} \vert \times 100 \qquad ( 11 )$$

I is the m×n reference image where K is the m×n generated image.

MATLAB has built-in functions for all of the above image quality metrics used. With regards to the MAPE metric calculated, “omitzero” and “omitnan” flags were set for cases in which the difference between the generated and reference image was zero and for when the reference pixel value is zero.

### 2.5 Model Training Information

The models were trained. The specific GPU nodes within the cluster used for training were running AMD 2× Epyc2 64 core CPUs with NVIDIA 2× V100 (32 GB) or AMD 2× Epyc3 64 core with NVIDIA 4× A100 (80 GB). The impact of different hyperparameters and model options was evaluated by establishing a base model with specific conditions and then varying the batch size, training schedule (#epochs), learning rate (LR), GAN mode, and normalization technique. A summary of the different model translations and training options can be found in Table 3.

TABLE 3
Model training and testing information for the various dataset manipulations investigated.

| | | Train/Test | Data | | |
|---|---|---|---|---|---|
| | Model | (# images) | Split | Dataset A | Dataset B |
| Image | Pix2Pix - Opt_8 | 2800/350 | split_data_quads | *polyhigh | *60 keV *[UL, UR, LL, LR] |
| Quadrants | Pix2Pix - Opt_8 | 2800/350 | split_data_quads | *polylow | *120 keV *[UL, UR, LL, LR] |
| K-fold | Pix2Pix - Opt_8 | 2800/700 | split_data_k[1-5] | polyhigh | polylow; [60, 80, 100, 120] keV |
| Cross | Pix2Pix - Opt_8 | 2800/700 | split_data_k[1-5] | polylow | polyhigh; [60, 80, 100, 120] keV |
| Validation | Pix2Pix - Opt_8 | ~2800/700 | split_data_k[1-5] | PH woTB | PL; [60, 80, 100, 120] keV - woTB |
| | Pix2Pix - Opt_8 | ~2800/700 | split_data_k[1-5] | PL woTB | PH; [60, 80, 100, 120] keV - woTB |
| DSR | Pix2Pix - Opt_8 | 1400/700 | split_data_k[1-5] | polyhigh | polylow |
| | Pix2Pix - Opt_8 | 700/700 | split_data_k[1-5] | polyhigh | polylow |
| PS | Pix2Pix - Opt_8 | 400/100 | split_data_k[1-5]_AP | polyhigh | polylow |
| (AP/LAT) | Pix2Pix - Opt_8 | 400/100 | split_data_k[1-5]_LAT | polyhigh | polylow |

Abbreviations:
PH = polyhigh;
PL = polylow;
woTB = without TwinBeam data;
UL = Upper Left;
UR = Upper Right;
LL = Lower Left;
LR = Lower Right;
DSR = Data Size Reduction;
PS (AP/LAT) = Projection Specific (Anterior-Posterior/Lateral)

The following training information was the default for the Pix2Pix GitHub implementation used and was unchanged for all the different model training-adam momentum term: 0.5, LR policy: linear, network initialization: normal, initialization scaling factor: 0.02, load size: 286, crop size: 256, and preprocess: resize and crop. The model checkpoints evaluated were saved in 5 epoch increments. Each model training starts from a polyhigh or polylow energy domain translating to the other polyenergetic domain and all virtual monoenergetic domains. The reason behind this is that in a clinical setting, the input to translate from is generally a polyenergetic x-ray acquisition.

By combining k-fold cross-validation with periodic test dataset evaluations, a comprehensive approach to model evaluation was adopted, ensuring robustness and reliability in results. This strategy allowed efficient use of the available data while maintaining a thorough assessment of the model's performance and generalization capability.

Model options and parameters were explored extensively to find the optimal settings for our experiments. Key parameters included initial learning rates, GAN modes, normalization techniques, and batch sizes. The best-performing model configuration, Pix2Pix-Opt_8, was identified through iterative optimizations:
Optimization 1: Varying one hyperparameter at a time.
Optimization 2: Fixing batch size, training schedule, and normalization technique, while varying learning rates and GAN modes.
Optimization 3: Extending training schedules with fixed batch size, learning rate, and normalization technique, while varying GAN modes.

Three GAN modes were explored: Vanilla GAN, Least-Squares GAN (ls-gan), and Wasserstein GAN with Gradient Penalty. The original GAN, Vanilla GAN, with cross-entropy loss, is known for instability issues. Least-Squares GAN uses least-squares loss for more stable training. Wasserstein GAN with Gradient Penalty (wgan-gp): Utilizes Wasserstein distance with a gradient penalty, offering stable training and reducing mode collapse.

The Pix2Pix-Opt_8 configuration utilized a batch size of 8, a learning schedule of 200 initial epochs with a subsequent 100 epochs for linear decay to zero, an initial learning rate of 0.0002, instance normalization, and the least squares GAN (ls-gan) mode.

### 2.5.1 Image Quadrants

To explore the impact of performing the domain translation within regions, models were also trained on images split into quadrants (256×256) after cropping to 512×512. These models used the split_data_quads data split. Cross-validation was not used in this instance.

### 2.5.2 Cross-Validation

K-fold cross-validation (k=5) was used due to the limited dataset size, ensuring generalizability and reliable performance estimates. The dataset was partitioned into 5 folds with an 80/20 training/testing split.

### 2.5.3 Dataset Size Reduction (DSR)

To assess the impact of dataset sizes, the original splits were further reduced to half and quarter sizes, maintaining the same testing dataset size (20%).

### 2.5.4 Projection-Specific (AP/LAT) Datasets

Datasets were reduced to specific projections (AP and LAT), resulting in smaller training (11.4%) and testing (2.85%) datasets, evaluated for the polyhigh to polylow domain translation.

### 2.5.5 TwinBeam Data Exclusion

The impact of excluding TwinBeam data was analyzed by comparing performance on datasets with and without TwinBeam data.

2.6 Model testing information

The same GPU nodes used for training were used if available. Otherwise, the standard and compute nodes were used for generating the test images. These nodes used AMD 2× Epyc2 64 core CPUs or AMD 2× Epyc3 64 core CPUs. The results were exported off the cluster partition to a secure restricted network location where a MATLAB script was used to calculate the model performance based on the metrics mentioned above.

### 3|Results

### 3.1 Evaluation Metrics

In the following, higher values represent better model performance for PSNR and SSIM, while lower values are better for MSE and MAPE.

### 3.2 Image Quadrants

Table 4 shows the model performance results when the datasets were split into quadrants. Only polyenergetic datasets were used for the two energy domain translations looked at. The results can be misleading at first glance. The PSNR, SSIM, and MSE show promising results for all quadrants and translations (polyhigh→60 keV; polylow→120 keV), but the MAPE for the polyhigh→60 keV translation is much higher than its counterpart.

TABLE 4
Quantitative evaluation of models trained using images that were split into quadrants.

| Data Split | Dataset A | Dataset B | Epoch | PSNR | SSIM | MSE | MAPE |
|---|---|---|---|---|---|---|---|
| split_data_quads | polyhigh - UL | 60 ke V - UL | 5 | 29.32 | 0.93 | 105.0 | 27.9 |
| | polyhigh - UR | 60 ke V - UR | 5 | 29.06 | 0.90 | 124.3 | 37.4 |
| | polyhigh - LL | 60 keV - LL | 5 | 29.83 | 0.93 | 117.7 | 25.2 |
| | polyhigh - LR | 60 ke V - LR | 5 | 29.93 | 0.90 | 148.8 | 25.3 |
| | polylow - UL | 120 keV - UL | 5 | 30.51 | 0.96 | 88.2 | 20.1 |
| | polylow - UR | 120 keV - UR | 5 | 30.03 | 0.95 | 102.1 | 18.1 |
| | polylow - LL | 120 keV - LL | 5 | 29.82 | 0.96 | 107.5 | 18.3 |
| | polylow - LR | 120 keV - LR | 5 | 29.74 | 0.96 | 128.4 | 16.3 |
| | polyhigh - UL | 60 ke V - UL | 300 | 32.10 | 0.96 | 53.4 | 21.8 |
| | polyhigh - UR | 60 ke V - UR | 300 | 31.23 | 0.93 | 72.5 | 26.7 |
| | polyhigh - LL | 60 keV - LL | 300 | 33.30 | 0.96 | 53.3 | 18.0 |
| | polyhigh - LR | 60 ke V - LR | 300 | 31.80 | 0.93 | 73.8 | 27.2 |
| | polylow - UL | 120 keV - UL | 300 | 34.16 | 0.98 | 43.0 | 12.3 |
| | polylow - UR | 120 keV - UR | 300 | 32.92 | 0.98 | 69.5 | 10.2 |
| | polylow - LL | 120 keV - LL | 300 | 33.05 | 0.98 | 80.1 | 11.9 |
| | polylow - LR | 120 keV - LR | 300 | 33.13 | 0.98 | 76.7 | 9.9 |

Abbreviations: UL = Upper Left; UR = Upper Right; LL = Lower Left; LR = Lower Right
Note:
The best-performing model results for a given metric are bold, underlined, and italicized.

The results can partially be attributed to the fact that a significant portion of the image is air/blank space around the body which the models can translate easily between. This heavily influences the metrics. The reason MAPE is higher for the polyhigh input versus the polylow input is likely to do with the scan field-of-view (SFOV) used in dual-source scanning for the Siemens Definition Flash. The polyhigh dataset comes from the B tube/detector combination which has a reduced number of detector elements and subsequent reduced FOV (330 mm) versus the A tube/detector FOV (500 mm).

This difference doesn't directly impact every image translation. However, for larger patients, anatomy outside of the B-FOV is not captured for the polyhigh data, but it is for polylow data due to the larger A-FOV. This issue is not seen for the centered and cropped 256×256 data that is used in the remainder of the model experiments, as it takes the center of the images which are not impacted by the different FOV or air/blank space in the peripheral of the images.

### 3.3 Cross-Validation & TwinBeam Data Exclusion

Table 5 shows the results for the cross-validation models with and without TwinBeam data.

TABLE 5
Quantitative evaluation of models trained using 5-fold cross-validation with and without TwinBeam data.

| Epoch | Data Split | Dataset A | Dataset B | PSNR | SSIM | MSE | MAPE |
|---|---|---|---|---|---|---|---|
| 300 | split_data_k | polyhigh | 60 keV | 27.26 ± 0.43 | 0.920 ± 0.012 | 196.5 ± 19.2 | 9.9 ± 0.4 |
| | [1-5] | polyhigh-woTB | 60 keV-woTB | 26.95 ± 0.34 | 0.916 ± 0.011 | 208.0 ± 26.7 | 10.2 ± 0.5 |
| | | polyhigh | 80 keV | 31.30 ± 0.19 | 0.959 ± 0.001 | 83.1 ± 5.9 | 5.7 ± 0.2 |
| | | polyhigh-woTB | 80 keV-woTB | 31.22 ± 0.30 | 0.961 ± 0.001 | 83.5 ± 6.7 | 5.7 ± 0.2 |
| | | polyhigh | 100 keV | 29.51 ± 0.32 | 0.963 ± 0.002 | 137.6 ± 14.9 | 7.2 ± 0.4 |
| | | polyhigh-woTB | 100 keV-woTB | 30.16 ± 0.39 | 0.966 ± 0.002 | 116.4 ± 14.9 | 6.4 ± 0.3 |
| | | polyhigh | 120 keV | 28.77 ± 0.36 | 0.957 ± 0.001 | 161.3 ± 18.6 | 7.7 ± 0.3 |
| | | polyhigh-woTB | 120 keV-woTB | 29.41 ± 0.42 | 0.960 ± 0.001 | 131.6 ± 14.6 | 6.7 ± 0.2 |
| | | polyhigh | polylow | 27.51 ± 0.54 | 0.923 ± 0.004 | 186.7 ± 23.9 | 9.3 ± 0.3 |
| | | polyhigh-woTB | polylow-woTB | 27.67 ± 0.42 | 0.925 ± 0.003 | 177.6 ± 20.8 | 9.2 ± 0.3 |
| | | polylow | 60 keV | 33.21 ± 0.38 | 0.961 ± 0.002 | 67.3 ± 7.1 | 5.3 ± 0.3 |
| | | polylow-woTB | 60 keV-woTB | 34.25 ± 0.38 | 0.971 ± 0.001 | 47.5 ± 4.7 | 4.6 ± 0.3 |
| | | polylow | 80 keV | 30.40 ± 0.39 | 0.963 ± 0.002 | 132.6 ± 17.7 | 7.3 ± 0.6 |
| | | polylow-woTB | 80 keV-woTB | 31.17 ± 0.24 | 0.965 ± 0.002 | 98.2 ± 10.7 | 6.3 ± 0.4 |
| | | polylow | 100 keV | 28.05 ± 0.58 | 0.947 ± 0.007 | 241.3 ± 44.5 | 9.4 ± 0.9 |
| | | polylow-woTB | 100 keV-woTB | 29.18 ± 0.32 | 0.948 ± 0.001 | 144.3 ± 19.2 | 7.3 ± 0.6 |
| | | polylow | 120 keV | 27.47 ± 0.36 | 0.943 ± 0.008 | 282.4 ± 46.7 | 10.1 ± 0.9 |
| | | polylow-woTB | 120 keV-woTB | 28.58 ± 0.27 | 0.940 ± 0.000 | 164.2 ± 17.5 | 7.9 ± 0.5 |
| | | polylow | polyhigh | 27.24 ± 0.42 | 0.935 ± 0.007 | 202.0 ± 26.3 | 10.0 ± 0.8 |
| | | polylow-woTB | polyhigh-woTB | 27.57 ± 0.22 | 0.935 ± 0.003 | 181.2 ± 17.2 | 9.7 ± 0.8 |

Abbreviations: woTB = without TwinBeam data
Note:
The best-performing model results for both with and without TwinBeam data for a given metric are bold, underlined, and italicized.

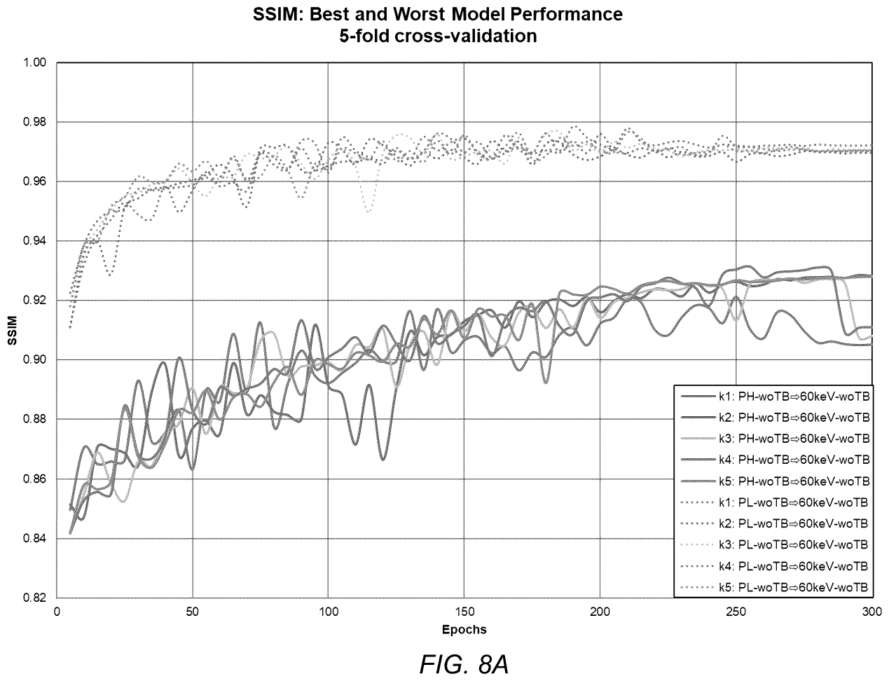

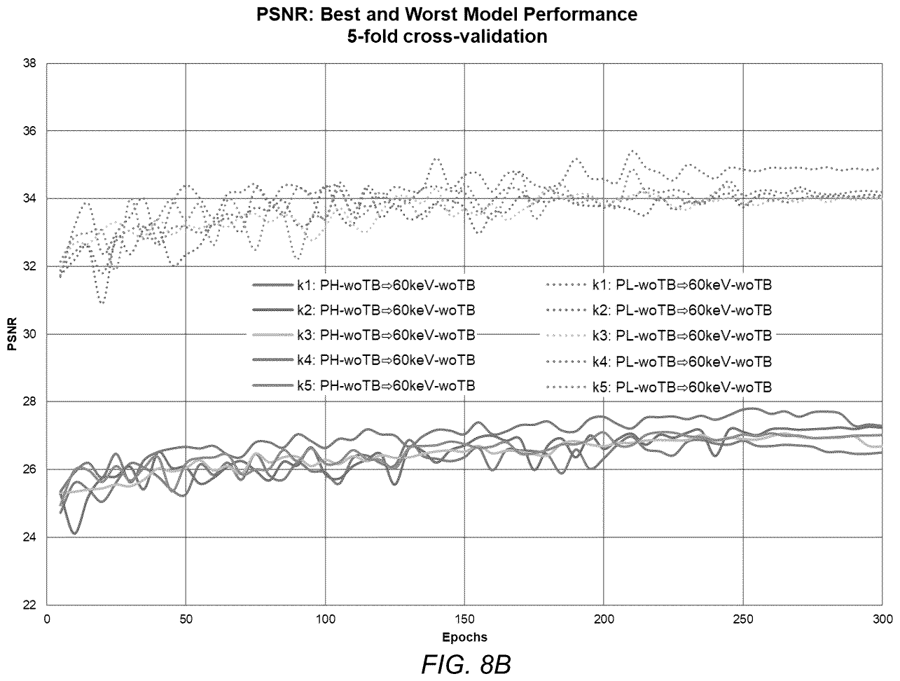

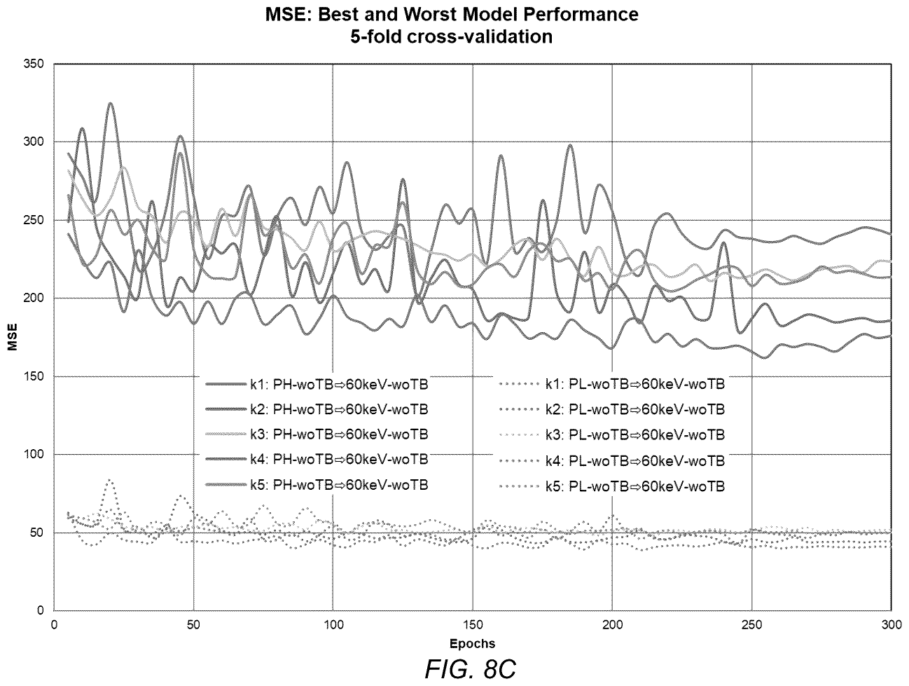

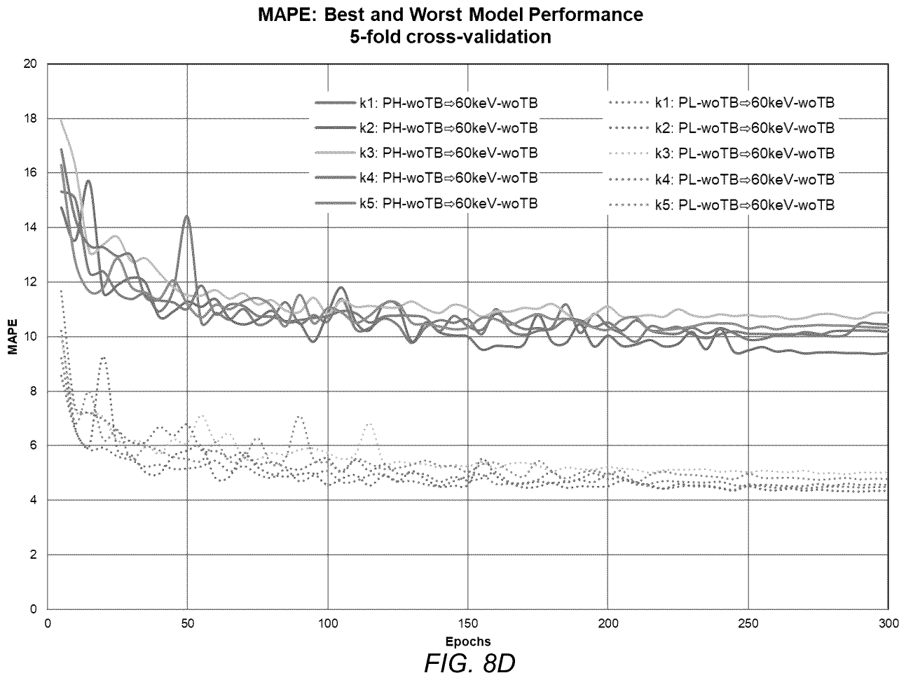

FIGS. 8A-8D show the polyhigh-woTB→60 keV-woTB (woTB =without TwinBeam data) and polylow-woTB→60 keV-woTB results for the different folds across all epochs. These were the worst and best-performing energy domain translations, respectively. FIGS. 8A-8D highlight the variability in results depending on the data splits used. It also shows when the model performance metrics start to stabilize and level off with minimal improvement.

The main goal of the cross-validation was to ensure that the different model performance metrics for the various energy translations were not tied directly to a particular training/test split. Some notable trends are seen across all the different energy domain translations. Generally speaking, the models trained on datasets without TwinBeam data performed better than their direct counterparts despite the reduction in dataset size. This indicates that there may be a benefit to training on datasets coming from a single dual-energy CT technology.

### 3.4 Dataset Size Reduction & Projection-Specific (AP/LAT) Datasets

Table 6 shows the results for the models that were trained on reduced dataset sizes and projection-specific datasets. Based on the results, the training dataset size has a small impact on model performance based on all the metrics used. It also indicates that using projection-specific datasets can improve model performance, as it had the smallest dataset size but was able to achieve comparable results. It outperformed the model trained on the largest dataset size on three of the four metrics used for the particular energy domain translation used for this sub-test.

TABLE 6
Quantitative evaluation of models trained using 5-fold cross validation with reduced dataset sizes or projection-specific datasets.

| | Train/Test | Data Split | Dataset A | Dataset B | Epoch | PSNR | SSIM | MSE | MAPE |
|---|---|---|---|---|---|---|---|---|---|
| K-fold Cross | 80/20 | split_data_k[1-5] | polyhigh | polylow | 5 | 26.11 ± 0.45 | 0.867 ± 0.002 | 217.8 ± 28.0 | 14.8 ± 1.5 |
| Validation | 40/20 | | | | | 26.02 ± 0.27 | 0.856 ± 0.006 | 222.8 ± 24.1 | 16.2 ± 1.7 |
| DSR | 20/20 | | | | | 25.69 ± 0.36 | 0.847 ± 0.007 | 239.4 ± 28.6 | 17.4 ± 1.6 |
| | 80/20 | | | | 300 | 27.51 ± 0.54 | 0.923 ± 0.004 | 186.7 ± 23.9 | 9.3 ± 0.3 |
| | 40/20 | | | | | 27.27 ± 0.40 | 0.917 ± 0.002 | 193.0 ± 25.4 | 9.4 ± 0.3 |
| | 20/20 | | | | | 26.93 ± 0.41 | 0.910 ± 0.001 | 203.2 ± 25.1 | 10.1 ± 0.2 |
| K-fold Cross | 11.4/2.85 | split_data_k[1-5]_AP | polyhigh | polylow | 5 | 26.42 ± 0.43 | 0.853 ± 0.003 | 198.5 ± 29.6 | 10.0 ± 1.0 |
| Validation | | split_data_k[1-5]_LAT | | | | 25.55 ± 0.59 | 0.795 ± 0.013 | 259.0 ± 47.3 | 23.7 ± 2.9 |
| PS (AP/LAT) | | split_data_k[1-5]_AP | | | 300 | 27.58 ± 0.47 | 0.911 ± 0.001 | 168.7 ± 25.9 | 8.5 ± 0.5 |
| | | split_data_k[1-5]_LAT | | | | 27.14 ± 0.51 | 0.908 ± 0.003 | 223.1 ± 31.9 | 12.3 ± 0.5 |

Abbreviations: woTB = without TwinBeam data; DSR = Data Size Reduction; PS (AP/LAT) = Projection Specific (Anterior-Posterior/Lateral)
Notes:
The values in the Train/Test column represent the percentage distribution of images from the original dataset allocated to the training and test datasets, respectively. The best-performing model results for the DSR and PS experiments for a given metric are bold, underlined, and italicized.

### 4|Discussions

This Example demonstrates the feasibility of an energy modulation network for projectional modulated radiography. The dataset manipulation, model optimization, and hyperparameter tuning were limited for this initial proof of concept Example but highlight the potential for improving model performance. Pix2Pix provided a starting point, but embodiments may employ different open-source models and/or proprietary models for supervised image-to-image translation problems in medical imaging.

The models trained on datasets with higher SSIMDOMAIN values (as shown in Table 7) had higher SSIM values and lower MAPE results, indicating that model performance is influenced by the similarity between the energy domains being translated. This suggests that the more similar the input and output energy domains are, the better the model performance will be.

TABLE 7
SSIM between energy domains.

| Data Split | Dataset A | Dataset B | SSIM_{DOMAIN} | |
|---|---|---|---|---|
| split_data_k[1-5] | polyhigh | 60 keV | 0.905 | ±0.002 |
| | polyhigh-woTB | 60 keV-woTB | 0.905 | ±0.003 |
| | polyhigh | 80 keV | 0.967 | ±0.001 |
| | polyhigh-woTB | 80 keV-woTB | 0.968 | ±0.002 |
| | polyhigh | 100 keV | 0.965 | ±0.002 |
| | polyhigh-woTB | 100 keV-woTB | 0.965 | ±0.002 |
| | polyhigh | 120 keV | 0.960 | ±0.002 |
| | polyhigh-woTB | 120 keV-woTB | 0.961 | ±0.002 |
| | polyhigh | polylow | 0.931 | ±0.002 |
| | polyhigh-woTB | polylow-woTB | 0.931 | ±0.002 |
| | polylow | 60 keV | 0.968 | ±0.002 |
| | polylow-woTB | 60 keV-woTB | 0.977 | ±0.001 |
| | polylow | 80 keV | 0.961 | ±0.001 |
| | polylow-woTB | 80 keV-woTB | 0.961 | ±0.001 |
| | polylow | 100 keV | 0.934 | ±0.001 |
| | polylow-woTB | 100 keV-woTB | 0.928 | ±0.001 |
| | polylow | 120 keV | 0.923 | ±0.001 |
| | polylow-woTB | 120 keV-woTB | 0.917 | ±0.001 |
| | polylow | polyhigh | 0.931 | ±0.002 |
| | polylow-woTB | polyhigh-woTB | 0.931 | ±0.002 |

Abbreviations: woTB = without TwinBeam data.

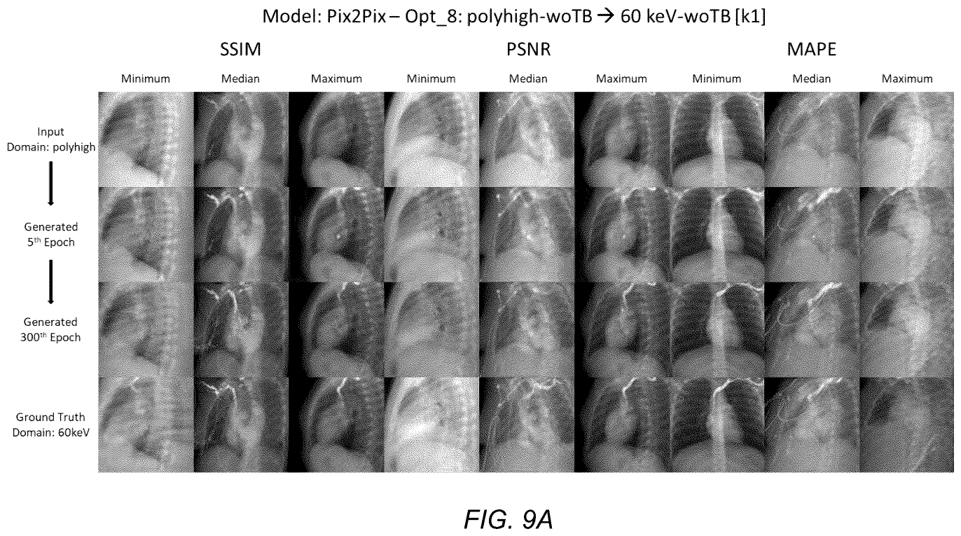

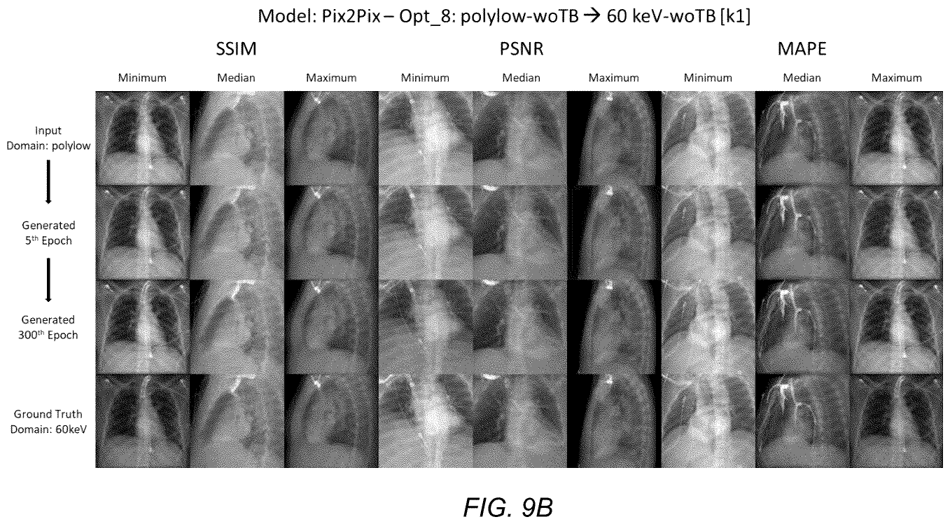

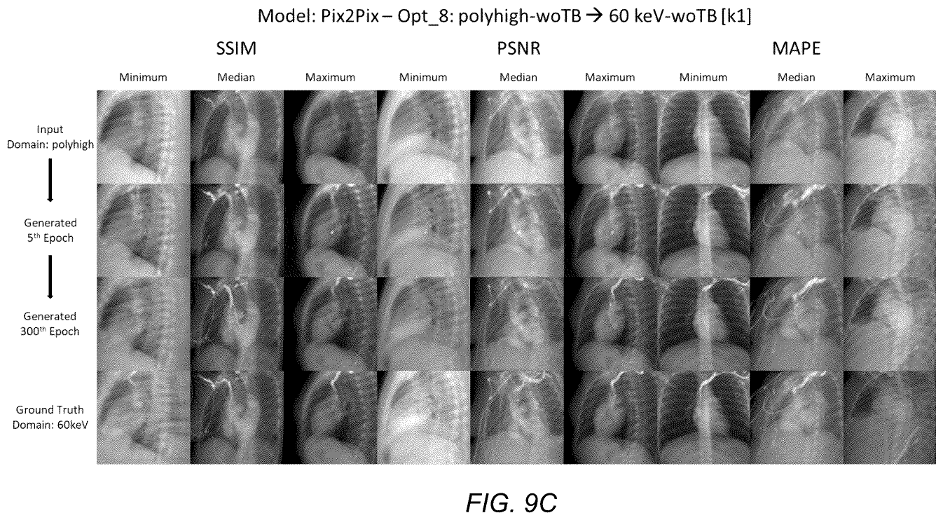

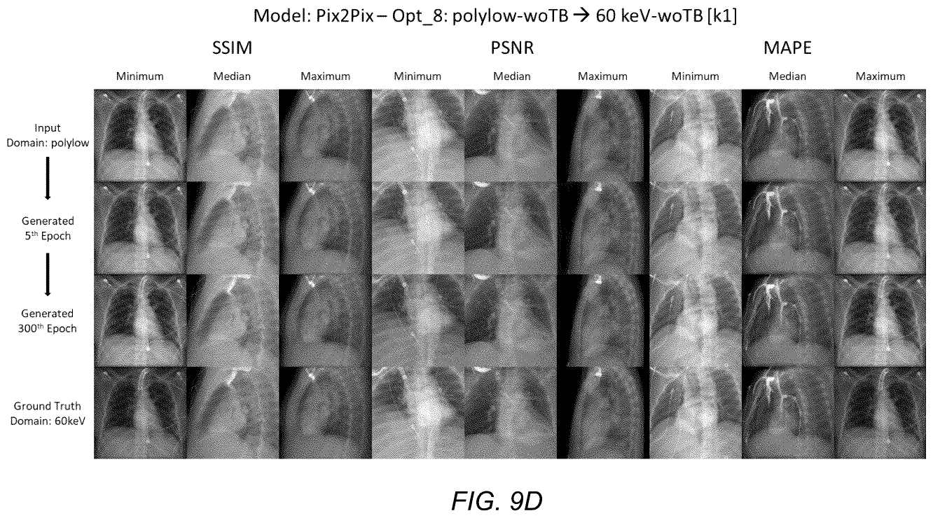

FIGS. 9A and 9B are images associated with the minimum, median, and maximum results for SSIM, PSNR, and MAPE for the worst performing model energy domain translations at the 300th epoch. For reference, the input image, the corresponding image at the 5th epoch, and the ground truth image are also provided.

FIG. 9C and 9D are images associated with the minimum, median, and maximum results for SSIM, PSNR, and MAPE for the best performing model energy domain translations at the 300th epoch. For reference, the input image, the corresponding image at the 5th epoch, and the ground truth image are also provided.

While exemplary embodiments of the present invention have been disclosed herein, one skilled in the art will recognize that various changes and modifications may be made without departing from the scope of the invention as defined by the following claims.

What is claimed is:

1. A method of digital modulated radiography for subsequent alignment of a subject who is to be subjected to radiation therapy or for diagnostic purposes, the method comprising
acquiring an image at a first polyenergetic energy;
translating at least part of the acquired image to a translated image of another polyenergetic or monoenergetic energy; and
outputting a generated image of the subject in which at least one region of interest (ROI) which is a sub-area and/or sub-volume of the generated image is based on the translated image, wherein the at least one region of interest (ROI) exhibits a difference of contrast with a neighboring region of the generated image, wherein the difference of contrast derives from a difference in one or more of image intensity, radiation intensity, and radiation energy, and wherein the at least one ROI exhibits continuity of subject anatomy with the neighboring region.

2. The method of claim 1, wherein the step of translating is performed by a machine learning model.

3. The method of claim 2, wherein the model is trained using paired datasets representing human anatomy at different energies.

4. The method of claim 3, wherein the paired datasets are from one or more photon counting detectors.

5. The method of claim 2, wherein the model comprises a conditional generative adversarial network (cGAN).

6. The method of claim 1, wherein the image at the first polyenergetic energy is a polyenergetic x-ray acquisition.

7. The method of claim 1, wherein the translating step comprises translating the acquired image to a lower energy domain than the first polyenergetic energy.

8. The method of claim 1, wherein the at least one region of interest (ROI) exhibits a discontinuity of display contrast with the neighboring region of the generated image in addition to a discontinuity of subject contrast with the neighboring region of the generated image.

9. The method of claim 1, wherein the acquired image is from a mammogram, wherein the method further comprises quantifying an amount of dense tissue using the generated image and/or estimating a percentage of breast density using the generated image.

10. The method of claim 9, further comprising tracking the amount of dense tissue and/or estimated percentage of breast density over time.

11. A method of radiation therapy, comprising aligning the subject using the image generated by claim 1; and
subjecting the aligned subject to radiation energy from radiation therapy equipment.

12. A non-transitory computer readable medium comprising computer program instructions which, when executed by one or more processors, cause the one or more processors to perform a method of digital modulated radiography for subsequent alignment of a subject who is to be subjected to radiation energy or for diagnostic purposes, the method comprising:
acquiring an image at a first polyenergetic energy;
translating at least part of the acquired image to a translated image of another polyenergetic or monoenergetic energy; and
outputting a generated image of the subject in which at least one region of interest (ROI) which is a sub-area and/or sub-volume of the generated image is based on the translated image, wherein the at least one region of interest (ROI) exhibits a difference of contrast with a neighboring region of the generated image, wherein the difference of contrast derives from a difference in one or more of image intensity, radiation intensity, and radiation energy, and wherein the at least one ROI exhibits continuity of subject anatomy with the neighboring region.
